(19) United States
(12) Patent Application Publication
(10) Pub. No.: US 2020/0356936 A1
(43) Pub. Date: Nov. 12, 2020

(54) SYSTEM AND METHOD FOR THE AUTOMATED INTERPRETATION OF OPERATIONAL DATA

(71) Applicant: eQstats Pty Ltd, Alexandria (AU)

(72) Inventor: Andrea Rodriguez, Alexandria (AU)

(21) Appl. No.: 16/938,669

(22) Filed: Jul. 24, 2020

Related U.S. Application Data
(63) Continuation of application No. PCT/AU2019/050053, filed on Jan. 24, 2019.

(30) Foreign Application Priority Data
Jan. 24, 2018 (AU) 2018200596

(51) Int. Cl. G06Q 10/06; G06F 40/56; G06F 16/904; G16H 40/20

(52) U.S. Cl. CPC G06Q 10/06393; G16H 40/20; G06F 16/904; G06F 40/56

(57) ABSTRACT

The present invention relates to a system and method for the automated interpretation of operational data and the automatic generation of reports. A system comprises of visualization and interpretation engine arranged to perform analysis and interpretation of operational data and generate reports. The plurality of feature brick modules are arranged to functionally interface with the visualization and interpretation engine to apply at least one logic scenario and generate textual content for the portion of the report and dependence of the output of the logic scenario.

### CROSS-REFERENCES TO RELATED APPLICATIONS

This application is a continuation application of International Patent Application No. PCT/AU2019/050053 entitled “SYSTEM AND METHOD FOR THE AUTOMATED INTERPRETATION OF OPERATIONAL DATA,” filed on Jan. 24, 2019, which claims priority to Australian Patent Application No. 2018200596, filed on Jan. 24, 2018, all of which are herein incorporated by reference in their entirety for all purposes.

### FIELD OF THE INVENTION

The present invention relates to a system and method for the automated analysis of operational data, and, particularly, but not exclusively, to a system and method for the automated analysis and interpretation of operational data in the healthcare industry.

### BACKGROUND

Monitoring of operational performance parameters for any industry is important. It is necessary to monitor operational indicators so that performance can be assessed, and if necessary, adjusted. Performance monitoring is an essential management tool. In some industries (for example, the health industry, the security industry, and others) monitoring and reporting aspects of operations may be a legal requirement.

Currently, there are many ways of monitoring performance, but the majority of them, particularly in industries where there is a lot of performance data generated (e.g. health services), require much manual handling.

In the health services industry for example, it is necessary to collect large amounts of data on various aspects of medical processes. Data may include incidences of visits of patients to a specialist, the outcome(s) of patient visits; procedure(s) undertaken for patient; effectiveness of those procedures; what processes were involved in the procedures; any other data relating to the procedure, patient, health professional, etc. Such data is required so that the effectiveness of the health service can be monitored, and if necessary, adjusted and improved. Monitoring may also be required for legal compliance and clinical decision making by a clinician, executive team, patient or guardian based on: the outcome of the historical and benchmarked data; c data identified precedent or potential risk; data likelihood/probability for future treatment or planned invention.

It will be appreciated that in industries such as the health services industry, there are many different types of processes and procedures. These generate extremely large amounts of data. All this data must be processed and analysed to a form suitable to enable meaningful interpretation and conclusion. For example, reports may be generated for management review, so that management can draw meaningful conclusions and take action. A patient may be scheduled to have elective heart surgery and wants to understand the probability re the operation success and potential risk based on the incidence and prevalence and historical performance by the institution as compared to other facilities.

There are a number of aspects to the monitoring of large amounts of complex data in sophisticated industries such as health services. Firstly, the data must be obtained. This may be done in many different ways. Data gathering can include input into computers via forms (e.g. on entering patient details into a procedure); surveys, for example post a medical process; entering data into databases, and many other data intake processes. Once the data is collected, it must generally be cleaned—there are usually many errors with manual input and collection of data.

Currently, there are a number of data collection methods and software tools that have been developed to facilitate entry of data, but the methods and tools do not provide an ideal solution.

Once the data has been collected (and usually stored in relatively large databases), it then has to be analysed and interpreted. Current analysis and interpretation requires significant manual input, although some tools are available to assist (e.g. general software tools such as Excel™ etc.). The manual resources required can be very large. The analysis and interpretation is extremely time consuming and comes at great cost. The manual requirement leads to some severe disadvantages:
bias in reporting—where humans are involved in reporting adverse outcomes, for example, based on performance and treatment of patients, there is the possibility of bias in the interpretation and reporting;
errors in reporting—many of the operatives involved in the interpretation and reporting may not be experts in this type of analysis and interpretation. In fact, many may be operatives in the industry and have no statistical skill or analytical knowledge to make assumptions about the data. For example, most nurses and administrators in the health industry have no skill or knowledge in the area of interpretation of data. There is a high risk, therefore, that errors are made;
the data may not be current—for example, most hospitals report on retrospective, old data, losing the opportunity to make process change on current data;
cost—the cost of the manual input required is immense.

Once the statistical analysis and interpretation has been carried out, then a report must generally be produced. Much manual preparation of the report is required. For example, providing text/words to accompany graphical displays is manually time consuming.

### SUMMARY OF THE INVENTION

In accordance with a first aspect, the present invention provides a system for the automated interpretation of operational data, comprising a computer having a processor, a memory and an operating system supporting computer processes, a visualisation and interpretation engine arranged to perform interpretation of the operational data and generate reports, a plurality of feature brick modules, each feature brick module being arranged to functionally interface with the visualization and interpretation engine to apply at least one logic scenario and generate at least a portion of a report, and to generate textual content for the portion of the report in dependence on the output of the logic scenario.

The term “interpretation” should be understood to cover the analysis and consideration of the data and may include the drawing of conclusions about the data.

At least an embodiment has the advantage that reports can be generated with minimal or no manual intervention for at least the interpretation of data and generation of the reports. In an embodiment, different textual content can be generated depending on the output of the logic scenario applied by the feature brick module.

In embodiments, textual content can include letters, words, numerals, any text.

In an embodiment, there are a plurality of feature brick module, each responsible for generating a respective portion of a report.

In an embodiment, at least one feature brick module may be responsible for generating the entire content of a report.

In an embodiment, at least one of the feature brick module may be arranged to apply interpretation of data. In an embodiment, at least one feature brick may be arranged to analyse data and produce graphical outputs for the report based on the data interpretation.

In an embodiment, the at least one feature brick module is arranged to operate on operational data in real time, changing outputs as data is interpreted and logic scenarios are implemented to dynamically affect the report.

In an embodiment, the system comprises at least one template, arranged to be populated by operation of the visualisation and interpretation engine and at least one feature brick, to form the report.

In an embodiment, the system further provides a data acquisition process, arranged to implement data acquisition. In an embodiment, the data acquisition process comprises an application interface arranged to interface with client systems, and which enables data from the client systems to be provided to the system in a form which the system can use for processing. In an embodiment, the at least one feature brick is arranged to obtain the data and place the data into required fields. In an embodiment, the at least one feature brick is arranged to “clean” the data.

Embodiments of the invention thus, advantageously, automatically analyse and interpret data obtained by the system. Advantageously, reports are output which provide meaningful information which can be used for performance review, amendment of processes and general management of the processes. All this is done automatically with minimal manual input.

In embodiments, this advantageously reduces bias and errors in reporting. In an embodiment, data can be processed “on the fly” as it is obtained by the system. The system can therefore provide “incident” reports as they occur or soon after they occur. This can result in much improved performance.

Further, embodiments of the present invention advantageously greatly reduce the cost of interpreting data, as minimal manual input is required.

In accordance with a second aspect, the present invention provides a method for automated interpretation of operational data, comprising the steps of, receiving operational data, implementing a visualisation and interpretation engine via a processor, the visualization and interpretation engine performing interpretation of the operational data and generating a report, and utilising at least one feature brick module to interface with the visualisation and interpretation engine and to apply at least one logic scenario and generate at least a portion of the report, generating textual content of the portion of the report in dependence on the output of the logic scenario.

In accordance with a third aspect, the present invention provides a computer program, comprising instructions for controlling a computer to implement a system in accordance with the first aspect of the invention.

In accordance with a fourth aspect, the present invention provides a non-volatile computer readable medium, providing a computer program in accordance with the third aspect of the invention.

In accordance with a fifth aspect, the present invention provides a data signal, comprising a computer program in accordance with the third aspect of the invention.

### BRIEF DESCRIPTION OF THE DRAWINGS

Features and advantages of the present invention will become apparent from the following description of embodiments thereof, by way of example only, with reference to the accompanying drawings, in which:

FIG. 1 is a high level schematic diagram illustrating operation of a system in accordance with an embodiment of the present invention;

FIG. 2 is a block schematic diagram of a system in accordance with an embodiment of the present invention;

FIG. 3 is a schematic diagram of a computing system that may be arranged to implement a system in accordance with an embodiment of the present invention;

FIGS. 4A and 4B are a flow diagram illustrating steps implemented by a system and method in accordance with an embodiment of the invention;

FIGS. 5A, 5B, 5C, and 5D illustrate a sample report generated by a system in accordance with an embodiment of the invention.

### DETAILED DESCRIPTION OF EMBODIMENTS OF THE INVENTION

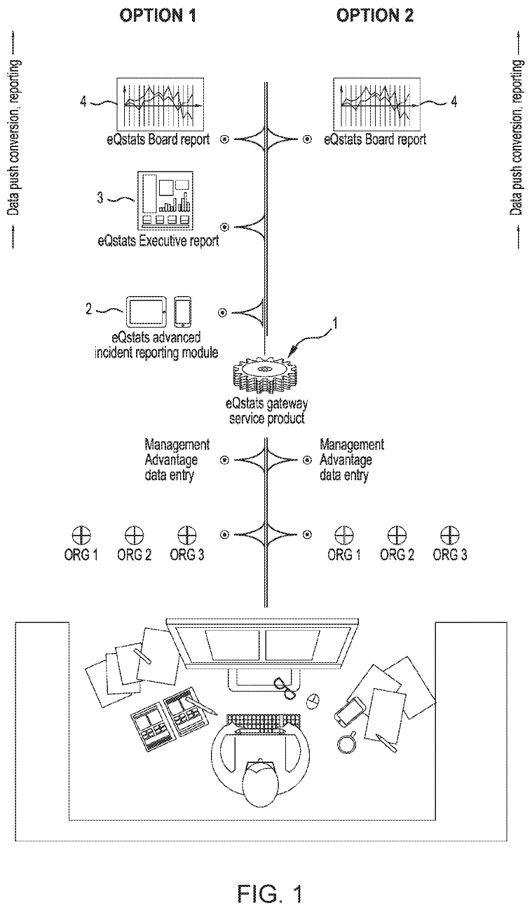

FIG. 1 is a high level diagram which illustrates how a system in accordance with an embodiment of the invention is arranged to obtain data and produce reports. Please note that terms shown in FIG. 1 (and other drawings) such as “eQstats” are trade names and trademarks and are not limiting to the scope of the present invention.

The system of this embodiment, generally designated by reference 1, is arranged for the processing of operational data relating to operations occurring in any industry or organisation. The system 1 is particularly arranged to facilitate application to statistical data for the health industry.

Statistical health data is obtained from any organisation (e.g. ORG1, ORG2, ORG3). The health data may be any health data. For example it may include incidences of visits to patients, procedures undertaken, effectiveness of procedures, patient transfer, patient accidents, illnesses, deaths, and more. There is a plethora of data available in the health industry, and much of it is regulated and required to be reported. Currently, the data may be obtained by many different pathways (health care, general practise, hospital, etc.) and processed in many different ways. Much of the processing to produce reports is manual, labour intensive and error prone, as discussed above.

In this embodiment, the data from ORG1, ORG2, ORG3 may be taken in via an AIP with the systems of ORG1, ORG2, ORG3. Alternatively or additionally the data may be manually input, or input in any other way.

The data is analysed by the system 1 and by the application of a plurality of feature brick modules which apply analysis and logic scenarios to the input data to interpret the data and build reports. This is done substantially without human intervention. Reports may be delivered to devices 2 (e.g. tablets, PCs, laptops, smartphones) too. Different types of reports may be provided by the system 1. Examples shown include an “Executive Report” 3 and Board Report 4.

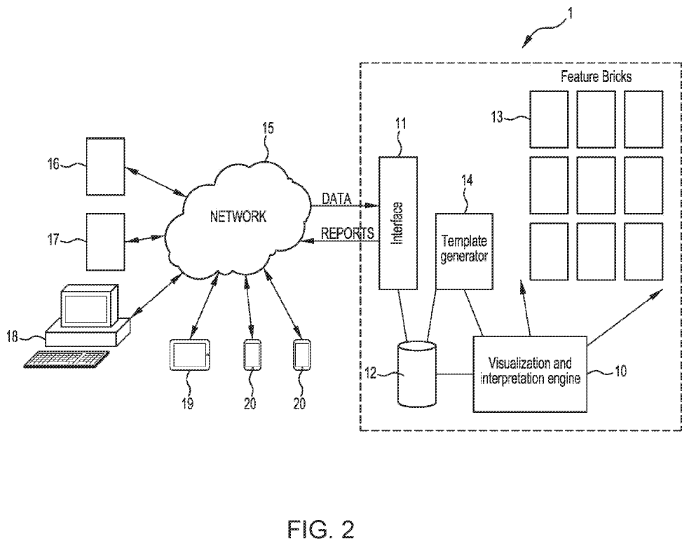

Referring to FIG. 2, an architecture of the system 1 of this embodiment will now be described.

The system 1 comprises a visualisation and interpretation engine 10 which is arranged to perform analysis and interpretation on operational data received via a system network interface 11 and which may be stored in database 12. A plurality of feature brick modules 13 are provided. There may be any number of these. Each feature brick is arranged to interface with the visualisation and interpretation engine 10 to interpret the operational data 11 and generate portions of a report to be generated. A plurality of feature bricks 13 may work to generate a single report, or, in embodiments, a single feature brick may generate a simple report. Feature bricks comprise software and instructions for the analysis and interpretation of data and the application of logic scenarios to the data. At least some of the feature bricks apply logic scenarios in order to generate wording for the report. Different wording is available for different results of the logic analysis. This facilitates automation of the wording for the generated reports. Similarly, feature bricks are responsible for graphical output of the report, depending on the logical analysis and statistical analysis of the data applied.

The system 1 also comprises a template generator 14 arranged, under the control of the visualisation and interpretation engine to generate templates to be populated by report content from the operation of the feature bricks 13. Different templates may provide the basis for different types of report.

The network interface 11 is arranged to receive operational data from remote systems via a network 15. Network 15 may be any telecommunications network, including the Internet. The systems may include hospital computing systems 16, 17, doctor surgery systems 18, mobile devices 19, 20 or any other remote devices. In embodiments the system 1 may be remote from systems from which it is receiving data. In other embodiments, the system 1 may be housed in an administration centre of an operations facility such as a hospital.

Data may be provided by being input manually via the systems or devices 16, 17, 18, 19, 20. Additionally or alternatively, APIs may be provided whereby the data is automatically uploaded to the system 1 from the systems or devices 16, 17, 18, 19, 20. In embodiments, remote apps may be supported by the systems and devices 16, 17, 18, 19, 20, which interface over the network 15 with the system to upload data.

Reports may be generated by the system 1 and loaded over the network 15 to the systems and devices 16, 17, 18, 19, 20.

Note there is no limitation on the types of systems or devices which may interface with the system 1 for the provision of data and the receiving of reports.

Reports may be updated in real time (“dynamically”). As data is uploaded to the system 1, it is processed by the system 1 and then reports which may already exist are amended in accordance with the processing. This means that reports can be updated “on the fly” in response to current data.

The system 1 may be implemented by any appropriate computing system on the basis of software supported by hardware.

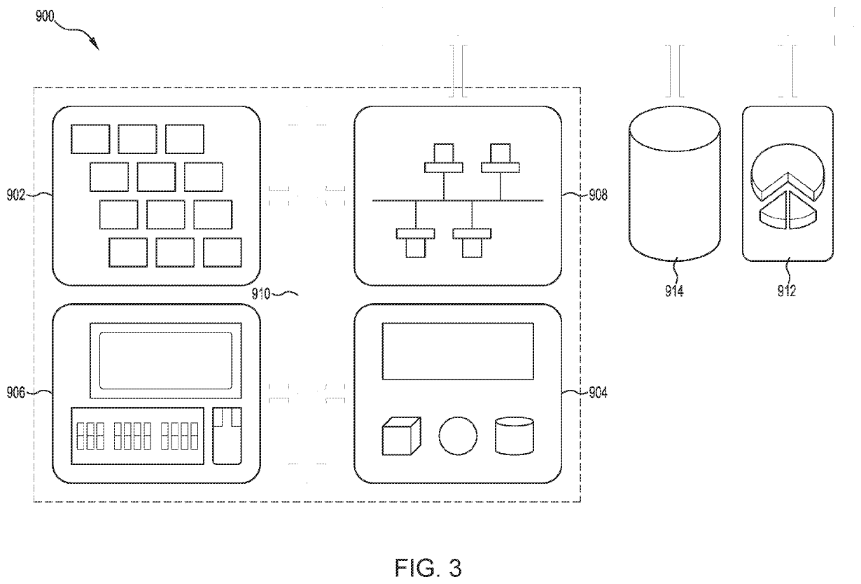

Referring to FIG. 3, the computer system 900 may be a high performance machine, such as a supercomputer, a desktop workstation or a personal computer, or may be a portable computer such as a laptop or a notebook or may be a distributed computing array or a computer cluster or a networked cluster of computers.

The computer system 900 also comprises a suitable operating system and appropriate software for implementation of embodiments of the present invention.

The computer system 900 comprises one or more data processing units (CPUs) 902; memory 904, which may include volatile or non-volatile memory, such as various types of RAM memories, magnetic discs, optical disks and solid state memories; a user interface 906, which may comprise a monitor, keyboard, mouse and/or touch-screen display; a network or other communication interface 908 for communicating with other computers as well as other devices; and one or more communication busses 910 for interconnecting the different parts of the system 900.

The computer system 900 may also access data stored in a remote database 914 via network interface 908. Remote database 914 may be a distributed database.

Computer processes are arranged to process program instructions and data in a known manner, to implement the system 1 of the present embodiment.

A computer system for implementing embodiments of the invention is not limited to the computer system described in the preceding paragraphs. Any computer system architecture may be utilised, such as standalone computers, networked computers, dedicated computing devices, handheld devices or any device capable of receiving processing information in accordance with embodiments of the present invention. The architecture may comprise client/server architecture, or any other architecture. The software for implementing embodiments of the invention may be processed by “cloud” computing architecture.

The computing system is provided with an operating system and various computer processes to implement functionality. The computer processes may be implemented as separate modules, which may share common foundations such as routines and sub-routines. The computer processes may be implemented in any suitable way and are not limited to separate modules. Any software/hardware architecture that implements the functionality may be utilised.

In more detail, this embodiment of the present invention provides a system which obtains operational data from an industry organisation, such as an organisation operating in the health industry e.g. a hospital, retirement home, etc. The system comprises a number of modules as discussed above in relation to FIG. 2, which implement the obtaining, analysis and interpretation of data, to produce outputs, such as reports, incident reports, board reports and other reports.

At a high level, the system implements a number of steps in the processing, analysis and interpretation of data in order to output reports which can be used to meaningfully deal with performance of the organisation. The steps include:

Step 1—obtaining data for use with the system. This may include utilising templates to organise the data. The data may include numeracy data and conversion of word weights e.g. risk scores of each incident into numerical data;

Step 2—the data is cleaned. Some data may be rejected;

Step 3—the data is analysed and interpreted by being subject to Bayesian statistical methodology;

Step 4—logic scenarios are implemented—there are some variables or further parameters set, like desired rate wanted to be achieved, risk score, sample size, confidence interval, benchmark comparison and historical results;

Step 5—using automated word selection and processing, output reports are generated via PDF dynamic built on the fly systems. Results are live and immediate.

The analysis and interpretation of the data, implementation of the logic scenarios and then output of report is implemented utilising the feature brick modules 13 interfacing with the visualisation and interpretation engine 10, to populate a template generated by template generator 14. Selection of bricks may depend on the template and report.

Each brick 3 represents the association of multiple data processing steps with the visualisation and interpretation engine 10.

Interpretation engine is software that takes the raw data, applying XSLT transforms (in this embodiment) to it to generate words that end up in the final report. The visualisation engine is software that takes the interpreted data and renders it as graphical output.

Each brick has a specific input format for the data it processes and can be provide as well formed XML (eXtensible Markup Language) or JSON (JavaScript Object Notation) as described below. The format is not limiting and could be adapted to any data structure that is capable of representing the required data elements.

Each brick provides a definition for the structure of the data required using an xml schema known as an XSD https://www.w3.org/XML/Schema. The xml schema defines input elements for all input data points required for processing at each subsequent step. Bricks may also make use of other external data sources for comparison to the input data.

An input stream of data is provided to the brick matching the required format. In the event data is not available directly in this format, pre-processing can be performed to convert to match the format required.

Each brick also has associated data processing mechanisms which performs validation and aggregation of the supplied data. External data sources may be utilised to assist in this validation phase.

A simple example of data is presented into both table and xml format, and also the matching schema is shown below:

| plot | xValue | yValue |
|---|---|---|
| row | 3.2 | 1.8 |
| row | 5.5 | 6.4 |

<plot>

<row><xValue>3.2<yValue>1.8</row>
<row><xValue>5.5<yValue>6.4</row>

</plot>
<xs:schema attributeFormDefault=“unqualified”
elementFormDefault=“qualified”
xmlns:xs=“http://www.w3.org/2001/XMLSchema”>

<xs:element name=“plot”>

<xs:complexType>

<xs:sequence>

<xs:element name=“row”
maxOccurs=“unbounded”

minOccurs=“0”>

<xs:complexType>

<xs:sequence>

<xs:element type=“xs:float”
name=“xvalue”/>
<xs:element type=“xs:float”
name=“yvalue”/>

</xs:sequence>

</xs:complexType>

</xs:element>

</xs:sequence>

</xs:complexType>

</xs:element>

</xs:schema>

Data cleansing and validation occurs as a transformational step between the input xml (data) and the rendered representation (output) of the data.

Data cleansing may reject outliers whereas validation may confirm that there is sufficient quantity of data presented such that valid statistical operations may be performed on the data set provided.

Each brick also generates a written report (html) to accompany the graphical and tabular representation of the data. The report is generated by executing a series of commands defined using XSLT (Extensible Stylesheet Language Transformations). XSLT is typically used to convert one form of XML to another, such as the rendering of an HTML (HyperText Markup Language) web page from input data.

The brick does not merely transform from the data from one form of XML to another; it performs analysis as defined by a clinical epidemiologist or statistician (when the embodiment is applied outside of the health industry), thereby enabling automation of this previously manual step.

A simplified example is to highlight each item in the result set where the value exceeded a threshold. This example uses the sample data provided in table 1.

<p>Epidemiological prologue</p>
<xsl:template match=“element/row”>
<xsl:variable name=“yValue” select=“yValue”/>
<xsl:choose>

<xsl:when test=‘$yValue>6’>
<p>Value presented exceeds 6.</p>

</xsl:when>
<xsl:otherwise>

<ext:Stratification( )/>

</xsl:otherwise>
</xsl:choose>
</xsl:template>

Extensions are also provided that may be called from within the XLST that allow for stratification subreports to be generated based on conditions found within the data. This is shown in the <xsl:otherwise> element of the previous XSLT definition for a brick. This allows for complex relationships to be defined and triggered only when appropriate conditions are found within the data.

Further stratification sub reports may also be triggered, allowing any level of nesting of sub reports as defined by the epidemiologist or statistician.

Examples of two feature bricks modules are given below. These may be many feature bricks and these are merely examples.

### Example 1

### Feature Brick ‘Flagged’ Results Child Events Statement Generated

Note—additional statements generated for some specific child event categories
Child event summary statement should not be generated until statistically valid
Child event summary statement cannot be produced unless there is a benchmark
Child event summary statement cannot be produced unless there is six months hx data
Child event summary statement cannot be produced unless there are denominators included for the months under study ie bed days

### BDD 010

Scenario: Statement generated for child event summary statement where six month rate is greater than benchmark rate, results are ‘flagged’
Given that the last six month performance percentile rate (n=166), 1.24% exceeds the child event external benchmark rate 0.92% by >0.32%
And there is a sample size >30 (n=166)
When I run the incident report
Then a statement is generated, External benchmark comparison for last six months data by category include falls rate of events being 0.32%>than benchmark rate.

### Example 2

### Feature Brick Statistically Significant Gain

Child event summary statement cannot be produced unless the sample size is statistically significant i.e = or >30
Child event summary statement cannot be produced unless there is a ‘gain’ recorded in the data table
Child event summary will be produced even if not an entire month of data in the current report
Child events summary statement will not be produced unless six month rate is lower than the benchmark rate

### BDD 009

Scenario: System generated child event summary statement occurs when the current report has achieved a statistically significant gain
Given that the falls rate of events has achieved (n=55) 2.56%
And there is a gain of 1.00% when compared to last six month report (n=183), 1.56%, result remains under the benchmark rate 1.98%
When I run the incident report
Then a statement is generated, Statistically significant result includes falls rate of events having increased by (n=55) 1.00% during this time period. The result remains below the benchmark rate.

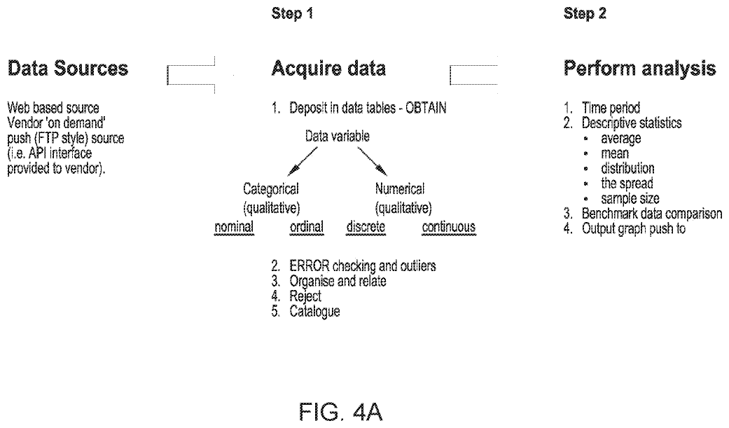

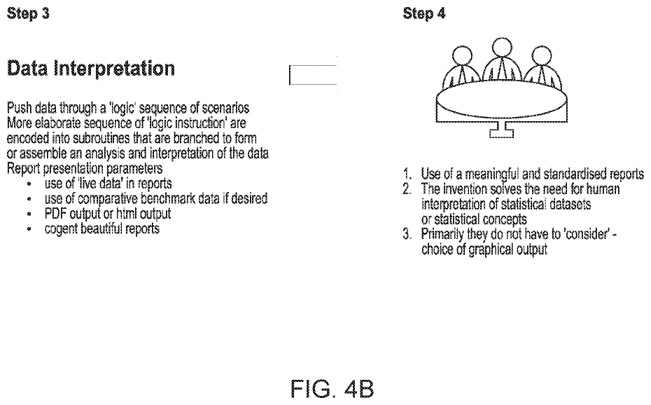

FIGS. 4A and 4B show a flow diagram illustrating steps implemented by the system of this embodiment. Data is obtained, as discussed above. The data is deposited in data tables at Step 1. The data is checked and cleaned.

At Step 2 analysis is performed as shown in the diagram. At step 3 logic sequences are applied to interpret the data. Analysis and logic sequences are applied by the brick modules that have been selected for the appropriate report. Reports are produced automatically and transmitted/updated to the recipient.

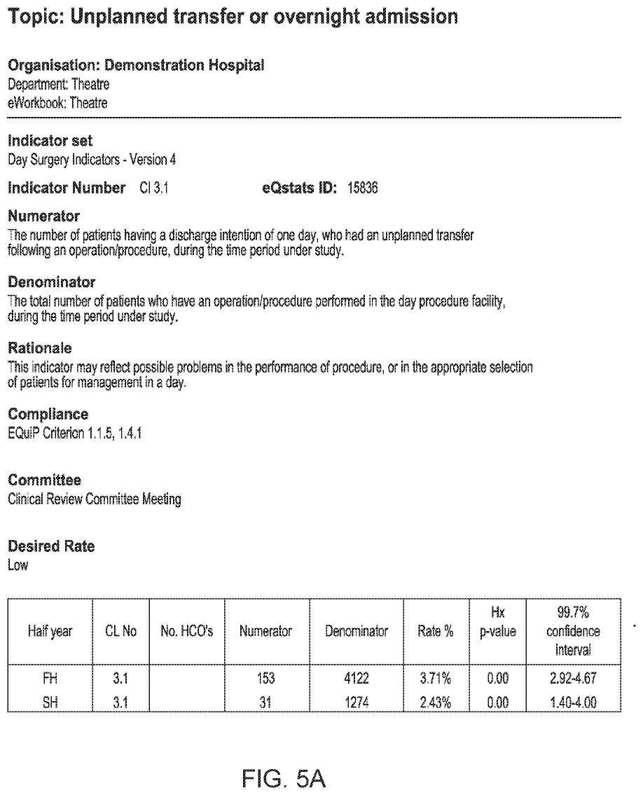

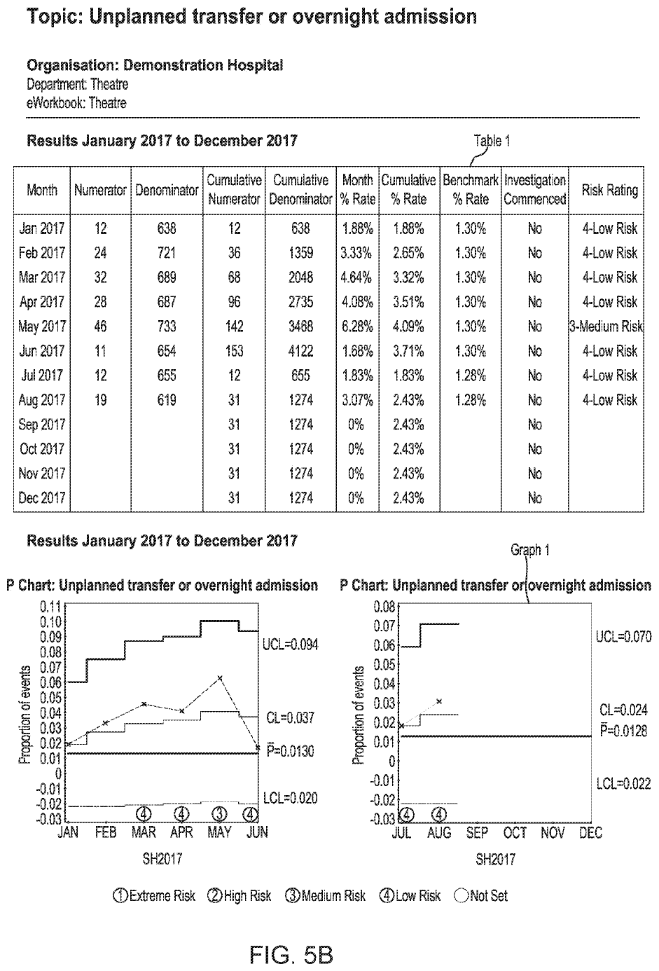

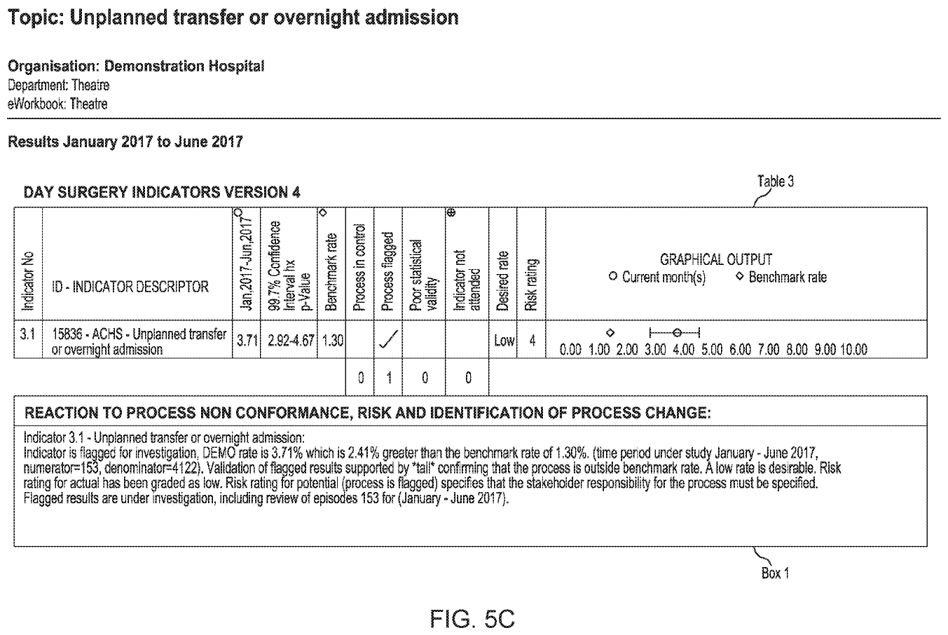

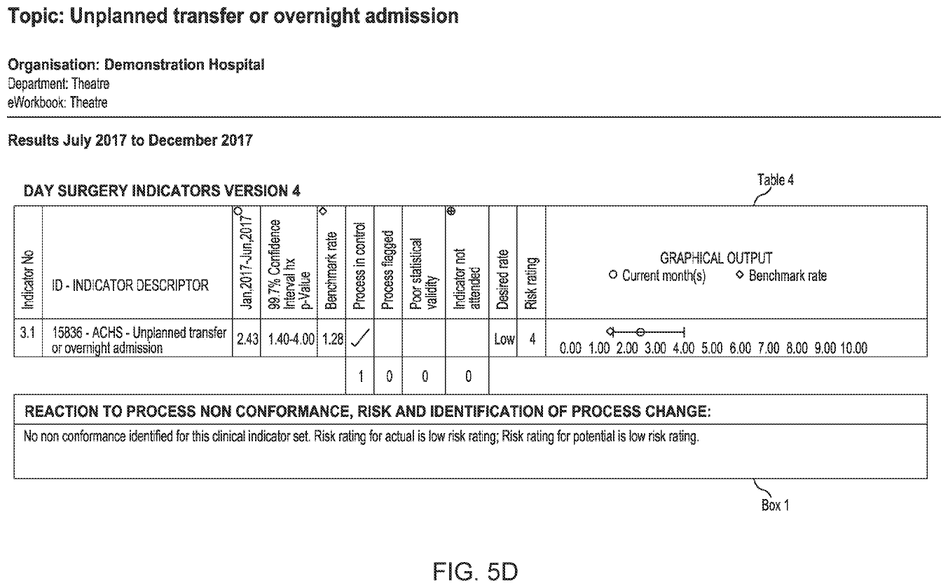

FIGS. 5A, 5B, 5C, and 5D illustrate an example report that may be generated by an embodiment of the invention.

The report includes a base template for the type of report. In this case “unplanned transfer or overnight admission” of a patient. The Numerator and Denominator of the data are loaded into the system (in this case the Numerator is the number of persons transferred overnight and the Denominator is the number of those in the hospital who are not transferred overnight).

The statistics are processed (see table 1) and brick modules which have performed the analysis also produce a graphical output (Graph 1).

Results are generated by the bricks (table 3 and table 4). In addition, the bricks are arranged to generate wording (box 1 and box 2), which is dependent on the results. In other words, the results are interpreted by the bricks logic and the appropriate wording is selected and generated.

Many different types of reports can be generated, depending on the processing applied by different bricks.

In the above embodiment, the system is applied in the health industry. The system is not limited to the health industry, but can be applied in any industry where statistical analysis and reports are required e.g. business, bureaucracy or any other industry.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

What is claimed is:

1. A system for automated interpretation of operational data, comprising a computer having a processor, a memory and an operating system supporting computer processes, a visualisation and interpretation engine arranged to perform interpretation of the operational data and generate reports, at least one feature brick module, each feature brick module accepting the operational data in an input format and being arranged to functionally interface with the visualization and interpretation engine to apply at least one logic scenario, said at least one logic scenario being associated with the feature brick module, to the accepted data, and generate at least a portion of a report, and to generate textual content for the portion of the report in dependence on whether the operational data satisfy the at least one the logic scenario.

2. A system in accordance with claim 1, wherein there are a plurality of feature brick modules, each responsible for generating a respective portion of a report.

3. A system in accordance with claim 1, wherein the at least one feature brick module is arranged to operate on operational data in real time, changing outputs as data is analysed and the logic scenarios are implemented to dynamically affect the report.

4. A system in accordance with claim 1, wherein at least one feature brick module is arranged to analyse data and produce graphical outputs for the report based on the data analysis.

5. A system in accordance with claim 3, wherein the at least one logic scenario associated with the feature brick module comprises executable instructions, arranged to be populated by the input operational data for execution.

6. A system in accordance with claim 1, further comprising a data acquisition process, arranged to implement data acquisition.

7. A system in accordance with claim 6 wherein the data acquisition process is arranged to obtain data from client systems, and wherein at least one feature brick is arranged to obtain the data and place the data into required fields.

8. A method for automated interpretation of operational data, comprising the steps of, receiving operational data, implementing a visualisation and interpretation engine via a processor, the visualization and interpretation engine performing interpretation of the operational data and generating a report, and utilising at least one feature brick module to interface with the visualisation and interpretation engine and to apply at least one logic scenario associated with the feature brick module to the operational data, and generate at least a portion of the report, generating textual content of the portion of the report in dependence on whether the operational data satisfy the logic scenario.

9. A method in accordance with claim 8, comprising the steps of utilising a plurality of feature brick modules to generate respective portions of a report.

10. A method in accordance with claim 8, comprising the step of operating on operational data in real time to change outputs as data is analysed to dynamically affect the report.

11. A method in accordance with claim 8, further comprising the step of acquiring data by applying one or more feature bricks to obtain the data and place the data into required fields.

12. A computer program, comprising instructions for controlling a computer to implement a system in accordance with claim 1.

13. A non-volatile computer readable medium, providing a computer program in accordance with claim 12.
